(19) United States
(12) Patent Application Publication
(10) Pub. No.: US 2025/0275704 A1
(43) Pub. Date: Sep. 4, 2025

(54) Printed circuit board (PCB)-needle assembly for minimally invasive and precise targeting specific areas in the brain of animals

(71) Applicant: Ludmila Gerashchenko, Chestnut Hill, MA (US)

(72) Inventor: Ludmila Gerashchenko, Chestnut Hill, MA (US)

(21) Appl. No.: 19/213,035

(22) Filed: May 20, 2025

Related U.S. Application Data
(63) Continuation-in-part of application No. 18/122,762, filed on Mar. 17, 2023.
(60) Provisional application No. 63/719,587, filed on Nov. 12, 2024.

(51) Int. Cl. A61B 5/262; A61B 5/00; A61B 5/293; A61B 5/296; A61N 1/05

(52) U.S. Cl. CPC A61B 5/262; A61B 5/293; A61B 5/296; A61B 5/6848; A61B 5/6868; A61N 1/0529; A61B 2562/166; A61B 2562/227

(57) ABSTRACT

A printed circuit board (PCB)-needle assembly designed for placement of cannulas and electroencephalogram (EEG) electrodes in small animals is disclosed. The assembly is connected to thin needles of multiple lengths, such that they can be placed into the brain at the chosen depth. It can be also soldered to a connector for enabling recordings of biopotentials. The design of the PCB-needle-connector assembly can be efficiently produced by specialized software. This software allows targeting any desired area(s) in the brain of a small laboratory animal based on the anteroposterior, mediolateral and dorsoventral coordinates of the animal's brain atlas. During the surgery, the assembly is placed on the head of a small animal and pressure is applied on it, so that the needles penetrate the bone. The bone can be drilled through or thinned to facilitate insertion of the needles. The needles can serve as EEG electrodes or used for placement of optical cannulas, or injecting test substances. The assembly is glued to the skull and may be additionally fastened by hooks on both sides of the skull. The procedure is fast and minimally invasive to the animal. It increases the accuracy and efficiency of research studies and improves animal welfare.

### TECHNICAL FIELD

Embodiments of the present disclosure relate to devices and methods of targeting specific areas of the brain of small laboratory animals. These devices and methods will be particularly useful for performing optogenetic studies, delivering test substances or sampling various fluids in the brain, and recordings of biopotentials and physiological parameters (electroencephalogram, electromyogram, brain temperature, blood flow, locomotion, and the like) in freely moving mice in both academic institutions and commercial laboratories.

### BACKGROUND

Generally, animals are used in scientific research to understand biomedical systems that lead to the development of useful drugs, therapies, and cures for diseases and pathologies. Mice are commonly used in biological research for multiple reasons, such as they are easily housed and maintained, they are relatively inexpensive, they reproduce quickly, their biological and behavior characteristics closely resemble those of humans, their various transgenic models are available, and the like. Approximately 20-30 million mice are used each year in the United States for biological and medical research. Mice are used in research much more than any other animals. For many experiments, it is imperative to surgically implant various devices into the mouse skull. Such implantations are particularly necessary in optogenetic studies. After being named “Method of the Year” in 2010 (PUBMED ID #21191368), optogenetic methods became widely used in research laboratories.

Despite optogenetic studies have been regularly performed by many researchers all over the world for more than two decades, they are still done using virtually the same surgical procedures that were originally developed. These procedures are both time-consuming and require specialized tools. They include injection of a vector that expresses optogenetic transgene into the brain region of interest followed by implantation of an optical cannula into the skull of a laboratory animal. The brain injection and cannula implantation are performed in anesthetized animals via a hole in the skull drilled in the desired location using a stereotaxic instrument. Electroencephalogram (EEG) electrodes are often implanted during these surgeries to directly assess brain activity during optogenetic stimulation of specific neuronal circuits. Most often screw EEG electrodes are used, which produce lesions in the cortex underlying the electrode positions. Such tissue damage results from screw insertion that exceeds the skull depth (average thickness of a mouse's skull across is less than 0.5 mm) and presses into the dura mater. If the dura mater and the cortex are injured, scar tissue (caused by neuroinflammation and gliosis) form around the electrode and may alter electrode impedance and EEG quality and have an impact on subsequent recordings even after extended recovery periods.

Further, the total duration for the implantation of cannulas and EEG electrodes in a laboratory animal typically requires at least 30-60 min even for a well-trained scientist. A long-lasting implantation procedure, particularly long-lasting narcosis, poses specific stress to the animal and leads to subsequent impairment of recovery with potential loss of the animal. Additionally, the cost associated with animal surgeries is high (stereotaxic instrument, surgical supplies, dental cement, screw electrodes, isoflurane, use of a surgical room, etc). Increasing time efficiency, simplifying surgical procedures for placing cannulas, and avoiding the brain damage produced by placement of screw EEG electrodes in small laboratory animals such as mice is highly desirable.

The present disclosure introduces novel means of implanting cannulas and EEG electrodes in small laboratory animals. We describe new efficient procedures for performing brain injections and placing optical cannulas and EEG electrodes in optogenetic and other research studies, which are based on the use of a printed circuit board (PCB). PCBs are inexpensive and can be manufactured within a few days at a very high degree of accuracy, such as dimensional tolerances are less than 0.1 mm. In our procedure, PCBs are generated and may be connected together. Generally, two or more needles are soldered to the PCBs-a needle for placing it at the bregma, and a needle for targeting the area of interest in the brain. The PCBs could be then easily positioned on the animal's skull by simply pushing one of the needles into the bregma while keeping one of the PCBs along the midline suture on the skull. During this procedure, another needle will get inserted into the skull at the exact location required by the investigator. PCBs are then glued to the skull, and the animal is allowed to recover from the surgery. Ear bar placing, measuring the distance from the bregma, skull drilling, and the use of stereotaxic instrument are not required in this procedure, which allows the investigator to complete the whole surgery in less than 10 minutes.

The procedure is fast and minimally invasive to the animal. It increases the accuracy and efficiency of optogenetic and other research studies and improves animal welfare. Thus, the present disclosure presents a novel research platform for research and development of clinical treatments of neurodegenerative and other diseases with great commercial potential.

### SUMMARY

An assembly containing PCBs, needles, and connectors designed for placement of cannulas and EEG electrodes in small animals and methods for their use are described. The assembly device allows performing optogenetic studies, delivering test substances or sampling various fluids in the brain, and recordings of EEG, EMG, brain temperature, blood flow, locomotion, and other physiological parameters from small animals including mice, rats and birds.

One aspect of an embodiment of the present invention provides the description of the PCB-needle device that is designed for precise placement of cannulas and EEG electrodes in small animals. The PCB is connected to thin needles of various lengths, so they could be placed into the brain at the chosen depth. PCBs are connected in a way so that their configuration would allow the needle insertion at the exact location on the animal's skull. When the assembled device is placed on the head of a small animal and pressure is applied toward the skull the pin electrodes penetrate the bone so that the device becomes fixed to the skull. It is then glued to the bone. Additionally, it could be fastened by hooks positioned at the sides of the skull. Very thin needles (30 gauge or less) can be used in this device as EEG electrodes, which do not produce any significant damage to the brain and thus allow a better-quality recording of biopotential signals from the brain. Additionally, LEDs can be added to the device to facilitate optogenetic studies.

Another aspect of an embodiment of the present invention provides the description of the method for placing PCB-needle devices on the top of the animal head. Currently, the placement of optical and infusion cannulas or EEG electrodes in a small animal involves surgical procedures that are invasive and time-consuming. We describe a method of the placement of cannulas and electrodes that can be done in less than 10 minutes. According to this method, the animal is briefly anesthetized with isoflurane or intraperitoneal injection of an anesthetic (e.g., ketamine-xylazine solution). The head of the animal is shaved, treated with antiseptic, and placed into a rubber holder that is used to make sure that the animal is not damaged during the device placement. Then, the PCB-needle-connector assembly is placed on the top of the animal head at the desired location. The selection of the location for the assembly placement could be easily done by placement of one of the needles at the bregma while keeping the PCB along the sagittal suture. A stencil containing holes for needle placement and skull landmarks (bregma, lambda, scull sutures) can be used to assist with precise selection of the desired location. To attach the assembly device to the animal head, pressure is steadily applied on the head stage toward the skull. Thinning of the bone or drilling it through could be used to facilitate insertion of the needles, especially if the needles are not sharp or the placement of the assembly is performed in bigger animals such as rats that have a thicker skull. The needles penetrate through the bone and stay fixed in the skull. Then the assembly device is glued to the skull, and the animal is returned to its home cage for recovery.

Designs of PCBs for targeting any desired area in the mouse brain can be generated by software. In such a case, when an investigator enters stereotactic coordinates of the animal atlas (i.e., anteroposterior, mediolateral and dorsoventral measurements) to the software, it will generate files containing designs of PCBs. These files can be read by software for electronic design automation (e.g., a freeware KiCad program), which could be then used to generate both 3D images of PCBs and Gerber files for PCB manufacturing.

Other aspects, embodiments and features of the system and method will become apparent from the following detailed description when considered in conjunction with the accompanying figures. The accompanying figures are for schematic purposes and are not intended to be drawn to scale. In the figures, each identical or substantially similar component that is illustrated in various figures is represented by a single numeral or notation. For purposes of clarity, not every component is labeled in every figure. Nor is every component of each embodiment of the device and method shown where illustration is not necessary to allow those of ordinary skill in the art to understand the device and method.

### BRIEF DESCRIPTION OF THE DRAWINGS

The preceding summary, as well as the following detailed description of the disclosed system and method, will be better understood when read in conjunction with the attached drawings. It should be understood, however, that neither the device nor the method is limited to the precise arrangements and instrumentalities shown.

FIGS. 1A-1C illustrate the use of printed circuit boards (PCBs) for targeting specific regions in a mouse brain, wherein a 26-gauge needle is soldered to one PCB for holding an optical cannula (FIG. 1A), a 30-gauge needle is soldered to another PCB for placement at the bregma (FIG. 1B), and the connected PCBs are secured to the mouse skull (FIG. 1C), with optional EEG/EMG recording components.

FIGS. 2A-2C illustrate the use of a printed circuit board (PCB) soldered to needles serving as EEG electrodes (FIG. 2A), which are inserted into the skull and fixed with dental cement (FIG. 2B), and a perspective view of the assembled PCB-needle device with two vertically oriented PCBs and a protruding needle (FIG. 2C), demonstrating the physical structure of the targeting assembly.

FIGS. 3A-3C illustrate the insertion of metallic hooks on the sides of the skull and their use to secure the PCB-needle assembly in place.

FIGS. 4A and 4B illustrate the placement of an LED between the PCBs to enable irradiation of the brain through the skull for use in optogenetic studies.

FIGS. 5A-5D illustrate the use of a silicone rubber holder during surgery, in which the animal's head is positioned to prevent injury while the PCB-needle assembly is inserted into the skull.

FIGS. 6A and 6B illustrate a PCB-needle assembly designed for targeting specific regions of the animal brain, showing the PCB components in a disassembled state (FIG. 6A) and in an assembled configuration (FIG. 6B).

FIGS. 7A-7C illustrate the use of a stencil for placement of needles in the desired location in the animal's skull.

### DETAILED DESCRIPTION

The following detailed description is provided to enable a person of ordinary skill in the art to make and use the embodiments of the present disclosure. Various modifications, substitutions, and variations will be apparent to those of ordinary skill in the art without departing from the scope of the disclosure. Reference is made to the accompanying drawings, which are provided for purposes of illustration and are not intended to limit the scope of the disclosure. It should be understood that the features illustrated and described with respect to one embodiment may be combined with features of other embodiments without departing from the spirit and scope of the present disclosure.

Throughout the drawings and the following description, like reference numerals may refer to similar or identical elements for clarity and consistency. The embodiments described herein relate to an assembly containing PCBs, needles, and connectors designed for placement of cannulas and EEG electrodes in small animals and methods for their use, as described in greater detail below.

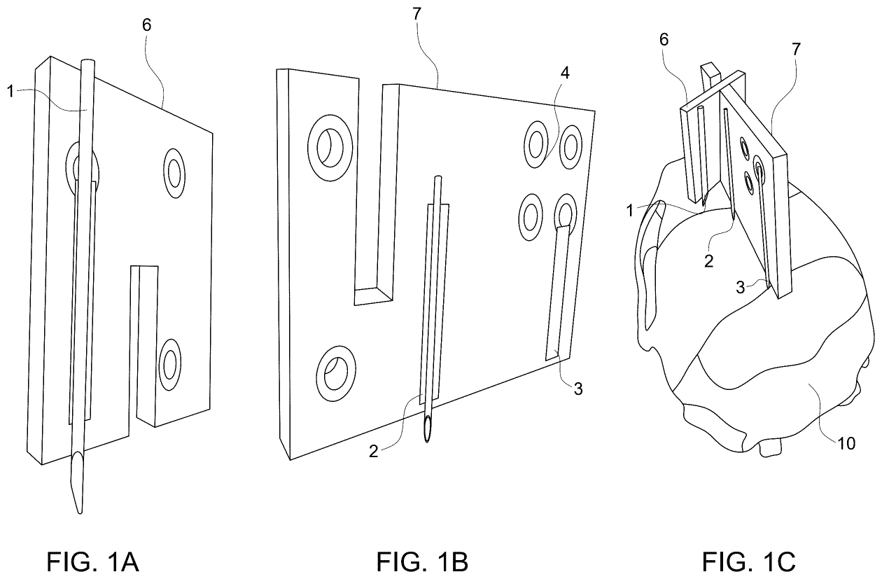

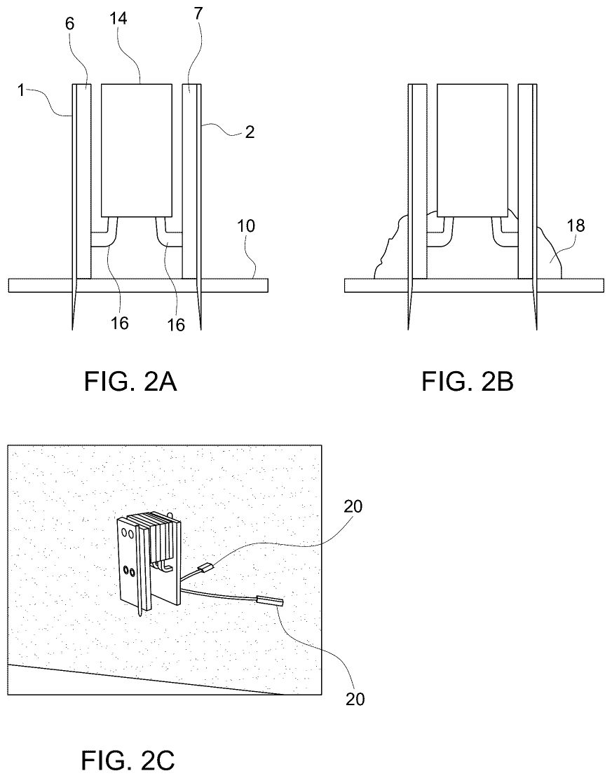

The present disclosure describes an assembly and method for precise implantation of cannulas and electrodes into the brains of small laboratory animals, including mice, rats, and birds. The assembly comprises one or more printed circuit boards (PCBs), such as first PCB 6 and second PCB 7, to which sharp needles, including a first needle 1 and a second needle 2, are connected. As shown in FIGS. 1A and 1B, the PCBs are configured such that the needles are positioned for accurate insertion into specific regions of the animal brain. For instance, the first needle 1 is soldered to the first PCB 6 for holding an optical cannula, while the second needle 2 is soldered to the second PCB 7 and placed at the bregma to guide positioning. EEG and EMG recording capabilities may be incorporated via additional needles 3 and 4 connected to the PCBs. When the assembled device is positioned on the skull 10 and downward pressure is applied, the needles penetrate the bone and anchor the device (FIG. 1C). Once inserted, the PCBs are glued to the skull using dental cement 18 (FIGS. 2A and 2B), and electrical connections are established using a connector 14 having a plurality of female contacts and wires 16. As further illustrated in FIG. 2C, EMG electrodes 20 can also be affixed to the device for additional physiological monitoring.

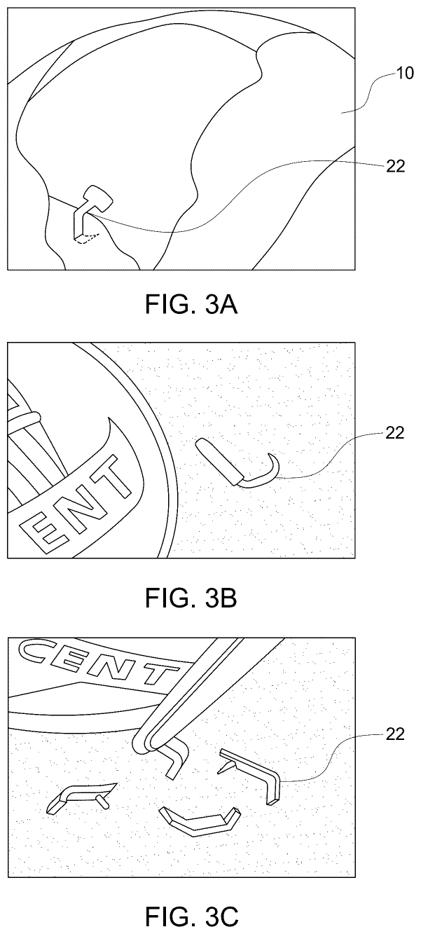

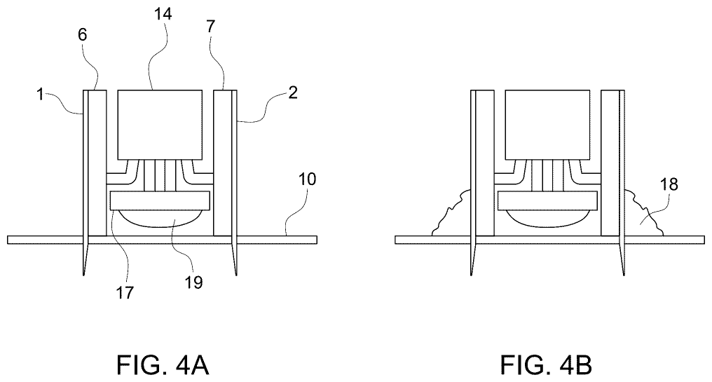

To enhance stability, metallic hooks 22 may be inserted into the sides of the skull 10, as seen in FIGS. 3A-3C, to secure the PCB-needle assembly in place during and after implantation. In some embodiments, optogenetic stimulation is enabled by the inclusion of an LED 17 and an optional lens 19 mounted between the PCBs, allowing light to be directed through the skull to the brain, as shown in FIGS. 4A and 4B.

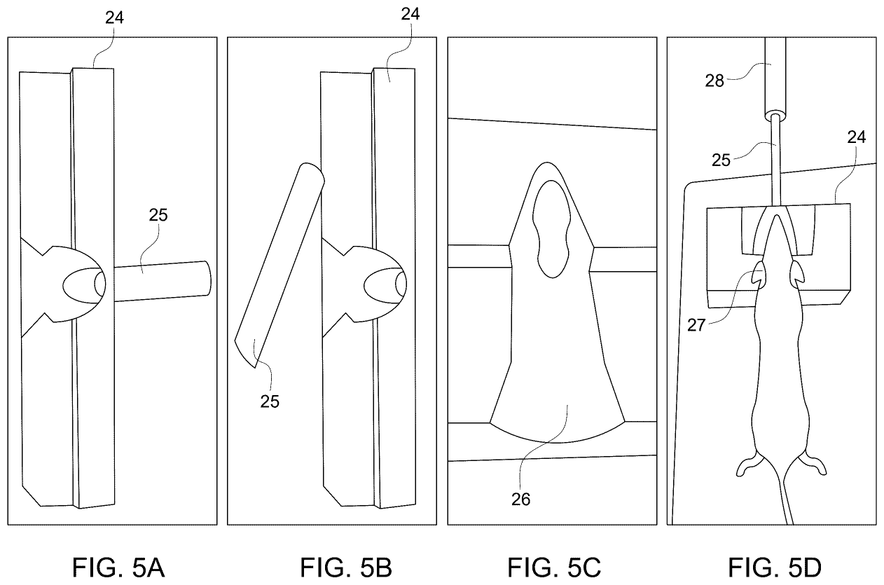

The procedure for implantation is designed to be rapid and minimally invasive. During surgery, the animal is anesthetized and positioned into a silicone rubber holder 24 (FIG. 5A), which ensures that the animal is not harmed while the PCB-needle assembly is pressed into the skull. A metallic hollow tube 25 is inserted into the rubber holder 24 to assist with stabilization, and can later be removed as shown in FIG. 5B. A recessed area 26 in the holder accommodates the head of the animal (FIG. 5C), which is then positioned such that the head 27 fits securely within the holder. In FIG. 5D, the animal receives anesthetic gas via the hollow metal tube 25 connected to a plastic hollow tube 28, while the device is inserted and fixed. Once in position, the device is glued to the skull using a light-curable cement, and the animal is injected with an analgesic, such as meloxicam, to promote pain-free recovery.

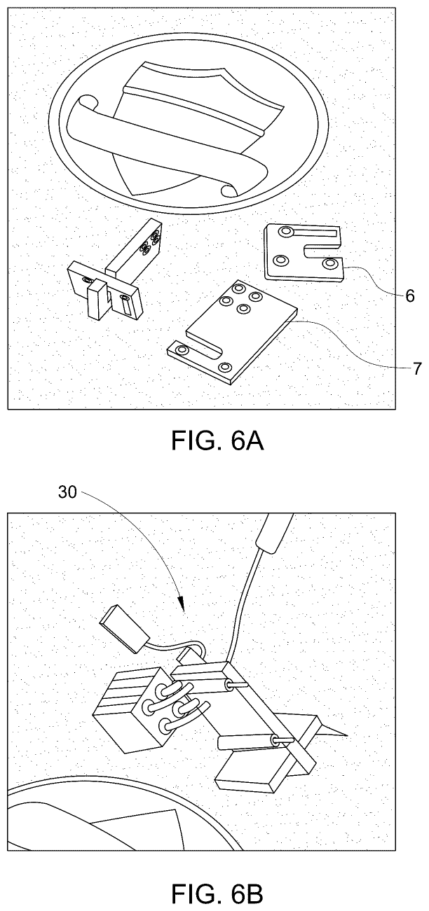

The design of the PCBs used in this system may be customized through the use of software, as illustrated in FIGS. 6A and 6B. By inputting stereotactic coordinates (anteroposterior, mediolateral, and dorsoventral) derived from an animal brain atlas, the software generates a custom PCB design that can be imported into a printed circuit design program, such as KiCad. These files may be rendered into 3D visualizations and Gerber files suitable for manufacturing. FIG. 6A shows PCBs 6 and 7 in a disassembled state, while FIG. 6B depicts the assembled PCB-needle device 30.

Collectively, the PCB-needle assembly and placement method offer a rapid, scalable, and precise alternative to traditional stereotactic surgery. The system supports a wide range of experimental applications including optogenetics, infusion or sampling of brain fluids, and high-fidelity recordings of EEG, EMG, brain temperature, blood flow, and locomotion data, among others. The invention simplifies the implantation process while enabling robust and reproducible targeting of brain structures in small animal models.

The needles used in the PCB-needle assembly may be solid, functioning solely as electrodes, or hollow, enabling both electrical recording and the insertion of optical fibers or the delivery and sampling of solutions, and in some embodiments, the assembly may include a combination of solid and hollow needles to support multiple functionalities simultaneously. The needle sizes may vary depending on the intended application, with a preferred narrow-gauge range of approximately 26-gauge to 30-gauge for minimal tissue disruption, and a broader acceptable range extending from about 22-gauge to 34-gauge to accommodate different implantation or recording needs.

In some embodiments, the PCB-needle assembly may use alternatives to traditional printed circuit boards, such as any rigid or semi-rigid insulating substrate capable of structurally supporting the placement of electrodes or cannulas while providing electrical connectivity through conductive traces or embedded wiring. These alternatives may include 3D-printed plastic or resin bases, ceramic carriers, flexible printed circuits, or other mechanically stable substrates with integrated conductive paths, allowing for similar functionality in aligning, supporting, and electrically connecting the needles.

The needles may be permanently affixed to the PCB, for example by soldering or adhesive bonding, or alternatively may be detachably connected to allow for modularity, replacement, or sterilization. In the detachable configuration, the PCB may include conductive female contacts or sockets (e.g., part of connector 14), into which needle-mounted male pins or conductive leads can be inserted and secured, allowing electrical continuity while enabling the needle component to be removed or replaced as needed. Alternatively, the PCB may include male connectors, and the needle assembly may incorporate corresponding female contacts to establish the electrical connection. Mechanical stabilization of detachable needles can be achieved through friction fit, snap-fit housing, threaded engagement, or the use of clamps or locking tabs integrated into the substrate or connector assembly.

The PCBs in the assembly may be positioned in various orientations depending on the application requirements; for example, they may be arranged parallel to each other, as shown in FIG. 2A, or perpendicularly, as illustrated in FIGS. 1A-1C, or in any other suitable configuration that ensures accurate targeting and secure placement of the needles relative to anatomical landmarks on the animal's skull.

As shown in FIGS. 1 through 6 and described in greater detail below, various embodiments of the present disclosure include systems and methods involving substrates with one or more electrically connected needles for implantation into the skulls of small laboratory animals, along with software-assisted design tools for generating substrate configurations based on stereotactic coordinates.

One aspect of the present disclosure provides a system for implantation in a laboratory animal, including a substrate and at least one needle attached to the substrate, where the at least one needle is electrically connected to a conductive pathway on or within the substrate and is configured to penetrate the skull of the animal and deliver an electrical signal to or receive an electrical signal from brain tissue.

In some embodiments, the substrate includes a printed circuit board (PCB). The at least one needle may be a solid needle configured to function as an electrode. In other embodiments, the at least one needle is a hollow needle configured to permit insertion of an optical fiber and/or fluid delivery or sampling. The needle may be permanently affixed to the substrate, for example by soldering or adhesive bonding. Alternatively, the needle may be removably attached to the substrate via a mechanical or electrical connector, such as a socket, snap-fit, or friction-fit assembly. The system may further include an LED mounted to the substrate and configured to emit light through the skull to stimulate neural tissue. The system may also include at least one electromyogram (EMG) electrode electrically connected to the substrate. The conductive pathway may include a soldered wire or printed conductive trace. The substrate may optionally include alignment features configured to position the needle at a stereotactically defined location relative to anatomical landmarks on the animal skull. In some implementations, the needles have a preferred gauge size in the range of approximately 26-gauge to 30-gauge, or a broader acceptable range extending from approximately 22-gauge to 34-gauge. The needle gauge may be selected based on the intended function of the needle, such that narrower gauges are used for EEG or EMG recordings and wider gauges are used for fluid delivery or fiber optic insertion.

In another aspect, a system for targeting brain regions in a laboratory animal includes a first printed circuit board (PCB) having at least one needle attached thereto, a second printed circuit board (PCB) having at least one needle attached thereto, and a connector electrically linking the first PCB to the second PCB, wherein the needles are configured to penetrate the skull of the animal, and at least one of the needles is electrically connected to a conductive pathway on one of the PCBs for recording or stimulation of brain activity. At least one needle in the system may be a hollow needle configured to allow passage of an optical fiber or fluid. The connector may include a multi-contact female socket configured to receive corresponding electrical leads from the needles, or alternatively may comprise a male connector configured to mate with female contacts on the needle assembly.

An LED may be positioned between the first and second PCBs and configured to irradiate the brain through the skull. At least one needle on each PCB may be used as an electroencephalogram (EEG) electrode. The system may also include at least one electromyogram (EMG) electrode electrically connected to the first or second PCB. The first and second PCBs may be arranged in a fixed geometric relationship to align the needles with stereotactic brain coordinates.

A method for implanting a needle into the brain of a laboratory animal using the system described above includes anesthetizing the animal, positioning the substrate carrying the at least one needle on the skull of the animal using anatomical landmarks, pressing the substrate so that the needle penetrates the skull, and securing the substrate to the skull using an adhesive. The method may further include placing the animal's head into a rubber holder during implantation to prevent injury. In some implementations, a light-curable cement is applied to bond the substrate to the skull after insertion. The method may also include attaching metallic hooks to the skull to provide additional mechanical stabilization of the substrate. Additionally, the method may include electrically connecting the needle to an external recording or stimulation system via a connector on the substrate.

The method may further include generating a substrate design by receiving stereotactic coordinates corresponding to a brain atlas of the animal, determining spatial positioning of one or more needles on the substrate based on the coordinates, and generating electronic design files for fabricating the substrate with needle-mount locations configured to target the specified brain regions. The electronic design files may include Gerber files compatible with printed circuit board manufacturing processes. A 3D visualization of the substrate and needle configuration may also be generated based on the received coordinates. The stereotactic coordinates may include anteroposterior, mediolateral, and dorsoventral measurements derived from a standardized animal brain atlas. The steps of receiving coordinates and generating the design files may be performed by a computing device executing instructions stored on a non-transitory computer-readable medium.

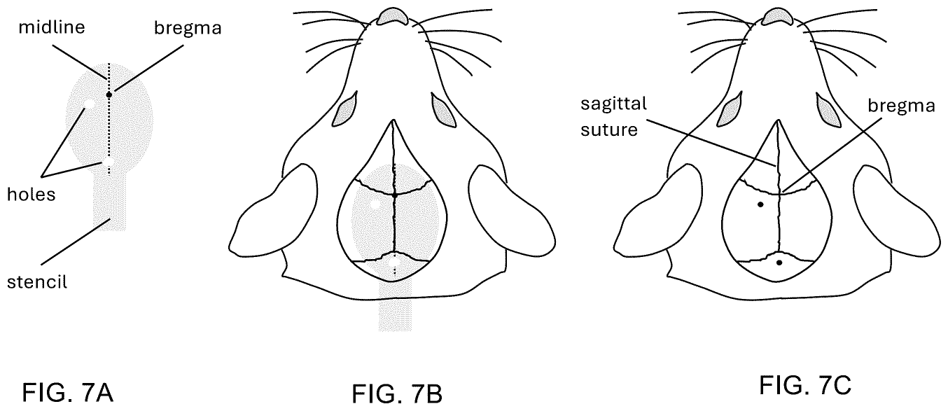

FIGS. 7A-7C illustrate the use of a stencil to facilitate accurate placement of the PCB-needle assembly on the skull of a laboratory animal. As shown, the stencil includes reference labels corresponding to anatomical landmarks such as bregma and the sagittal suture (midline). In use, the stencil is aligned with the animal's skull so that bregma and the sagittal suture coincide with the labeled regions on the stencil. Once aligned, the desired insertion points for the needles are marked directly onto the skull using a marker through designated holes in the stencil. To aid subsequent penetration by the needles, the marked bone locations may optionally be thinned or drilled, allowing for smoother and more precise insertion of the PCB-needle assembly.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

Although the invention is described herein with reference to specific embodiments, various modifications and changes can be made without departing from the scope of the present invention as set forth in the claims below. Accordingly, the specification and figures are to be regarded in an illustrative rather than a restrictive sense, and all such modifications are intended to be included within the scope of the present invention. Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements.

The foregoing detailed description is merely exemplary in nature and is not intended to limit the invention or application and uses of the invention. Furthermore, there is no intention to be bound by any expressed or implied theory presented in the preceding technical field, background, brief summary, or the following detailed description.

What is claimed is:

1. A system for implantation in a laboratory animal, comprising:
a substrate; and
at least one needle attached to the substrate,
wherein the at least one needle is electrically connected to a conductive pathway on or within the substrate and is configured to penetrate a skull of the animal and deliver an electrical signal to or receive an electrical signal from brain tissue.

2. The system of claim 1, wherein the substrate comprises a printed circuit board (PCB).

3. The system of claim 1, wherein the at least one needle is a solid needle configured to function as an electrode.

4. The system of claim 1, wherein the at least one needle is a hollow needle configured to permit insertion of an optical fiber and/or fluid delivery or sampling.

5. The system of claim 1, wherein the at least one needle is permanently affixed to the substrate.

6. The system of claim 1, wherein the at least one needle is removably attached to the substrate via a mechanical or electrical connector.

7. The system of claim 1, further comprising an LED mounted to the substrate and configured to emit light through the skull to stimulate neural tissue.

8. The system of claim 1, further comprising at least one electromyogram (EMG) electrode electrically connected to the substrate.

9. The system of claim 1, wherein the conductive pathway comprises a soldered wire or printed conductive trace.

10. The system of claim 1, wherein the substrate includes alignment features configured to position the needle at a stereotactically defined location relative to anatomical landmarks of the animal skull.

11. A system for targeting brain regions in a laboratory animal, comprising:
a first printed circuit board (PCB) having at least one needle attached thereto;
a second printed circuit board (PCB) having at least one needle attached thereto; and
a connector electrically linking the first PCB to the second PCB,
wherein the needles are configured to penetrate the skull of the animal, and at least one of the needles is electrically connected to a conductive pathway on one of the PCBs for recording or stimulation of brain activity.

12. The system of claim 11, wherein at least one needle is a hollow needle configured to allow passage of an optical fiber or fluid.

13. The system of claim 11, wherein the connector comprises a multi-contact socket configured to receive corresponding electrical leads from the needles.

14. The system of claim 11, further comprising an LED positioned between the first and second PCBs and configured to irradiate the brain through the skull.

15. The system of claim 11, wherein at least one needle on each PCB is used as an electroencephalogram (EEG) electrode.

16. The system of claim 11, further comprising at least one electromyogram (EMG) electrode electrically connected to the first or second PCB.

17. The system of claim 11, wherein the first and second PCBs are arranged in a fixed geometric relationship to align the needles with stereotactic brain coordinates.

18. The system of claim 11, wherein the needles have a gauge size in a preferred range of approximately 22-gauge to 34-gauge.

19. A method for implanting a needle into the brain of a laboratory animal using the system of claim 1, the method comprising:
anesthetizing the animal;
positioning the substrate carrying the at least one needle on the skull of the animal using anatomical landmarks;
pressing the substrate so that the needle penetrates the skull; and
securing the substrate to the skull using an adhesive.

20. The method of claim 19, further comprising placing the animal's head into a rubber holder during implantation to prevent injury.

21. The method of claim 19, further comprising applying a light-curable cement to bond the substrate to the skull after insertion.

22. The method of claim 19, further comprising attaching metallic hooks to the skull to provide additional mechanical stabilization of the substrate.

23. The method of claim 19, further comprising electrically connecting the needle to an external recording or stimulation system via a connector on the substrate.

24. The method of claim 19, further comprising generating a substrate design by receiving stereotactic coordinates corresponding to a brain atlas of the animal, determining spatial positioning of one or more needles on the substrate based on the coordinates, and generating electronic design files for fabricating the substrate with needle-mount locations configured to target the specified brain regions.

25. The method of claim 24, wherein the stereotactic coordinates comprise anteroposterior, mediolateral, and dorsoventral measurements derived from a standardized animal brain atlas.

26. The method of claim 19, wherein the desired location for placement of needles in the skull is assisted by a stencil.

27. The method of claim 19, wherein the bone is thinned or drilled through to make it easier for the needles to penetrate via the skull.

28. A system for targeting brain regions in a laboratory animal, including a first printed circuit board (PCB) having at least one needle mounted thereon, a second printed circuit board (PCB) having at least one needle mounted thereon, and a connector electrically linking the first and second PCBs, wherein the spatial arrangement between the first and second PCBs and the positioning of the needles on each PCB are determined based on stereotactic coordinates or anatomical landmarks of the animal brain, such that the needles are configured to penetrate the skull at locations corresponding to specific brain regions for delivery, stimulation, recording, or sampling.
